(19) United States
(12) Patent Application Publication
(10) Pub. No.: US 2015/0218502 A1
(43) Pub. Date: Aug. 6, 2015

(54) FLUIDIC DEVICE FOR STUDYING OF SURFACE-DWELLING MULTICELLULAR LAYERS AND MICROBIAL BIOFILMS

(71) Applicant: UNIVERSITE CATHOLIQUE DE LOUVAIN, Louvain-la-Neuve (BE)

(72) Inventors: Thomas Vanzieleghem, Perwez (BE); Jacques Mahillon, Nil-Saint-Vincent (BE); Hervé Jeanmart, Jambes (BE); Simon Degand, Bruxelles (BE); Christine Dupont, Ottignies (BE); Sandy Ladeuze, Lesve (BE)

(21) Appl. No.: 14/426,177

(22) PCT Filed: Sep. 6, 2013
(86) PCT No.: PCT/EP2013/068427

§ 371 Date: Mar. 5, 2015

(30) Foreign Application Priority Data
Sep. 6, 2012 (EP) 12183279.4

(51) Int. Cl. C12M 3/06; C12Q 1/02; C12M 1/12; C12M 1/00

(52) U.S. Cl. CPC C12M 23/16; C12M 23/22; C12Q 1/02; C12M 23/40; C12M 25/04

(57) ABSTRACT

The present invention relates to a fluidic device that can be used for the analysis of surface-dwelling multicellular layers, some of which comprise biofilm and can be referred to as biofilms, and their formation under controlled dynamic conditions. More particularly, the surface in the fluidic chamber on which the multicellular layer is grown is detachable and/or removable from the fluidic chamber, thereby providing a highly versatile device.

### FIELD OF THE INVENTION

The present invention relates to a fluidic device that can be used for the analysis of surface-dwelling multicellular layers and their formation under controlled dynamic conditions. More particularly, the surface in the fluidic chamber on which the multicellular layer is grown is detachable and/or removable from the fluidic chamber, thereby providing a highly versatile device.

### BACKGROUND

Surface dwelling multicellular layers such as for instance Biofilms are complex communities enmeshed in a self-produced matrix. In nature, biofilms represent about 90% of the bacterial lifestyle since it offers protection against all types of environmental threats (e.g. antibiotics, sanitizing agents). Biofilms are also intimately bound to human activities with both desired (wastewater treatment and gasification) and undesired aspects (corrosion, food intoxication or chronic infections). Biofilm research has gained increasing attention over the past years and this trend will certainly continue in the following years.

For observing and monitoring cellular biological processes (e.g. biofilms), cell-based assays are required where the behavior of living cells and changes in cell behavior in response to various conditions and agents may be observed, for instance through imaging techniques (visualization of microscopic processes through the use of agents that provide detectable signals representative of cellular components or events). Typically, these observations are performed in microtiter plates that contain the cells that can be observed through a microscope. However, these stationary conditions only offer a very narrow range of settings to observe biofilms and hence they do not provide a realistic image when it comes to monitoring biofilms because in nature these systems are usually formed in more dynamic environments, which require simulation of the actual real-life situations.

Formation and monitoring of a multicellular layer is a complex process where many factors (e.g. microorganisms, fluid flow and type, surface material and structure) play a role. Therefore, it is critical to perform the growth of multicellular layers in a versatile device that allows all these factors to be included into the simulation.

In view of the foregoing, there is a need for devices to perform assays, such as biochemical and/or cellular assays that provide for flow of fluid throughout the device so that nominally adhering substances, such as the cells in the multicellular layers can be monitored in in vitro conditions close to actual real-life situations.

While several tools enabling the analysis and study of multicellular layers are available, there seems to be a need for tools that act in a more accurate and more versatile manner.

### SUMMARY OF THE INVENTION

The present invention relates to a fluidic device for cultivation and/or analysis of surface dwelling multicellular layers comprising:
a fluidic chamber comprising one or more surfaces enclosing the fluidic chamber through which fluid can flow along a flow path, wherein said fluidic chamber is characterized by having a Young's Modulus [E] ranging between 500 kPa and 5 MPa, preferably ranging between 500 kPa and 2 MPa, more preferably ranging between 500 kPa and 1 MPa;
an inlet port fluidically connected to said fluidic chamber;
an outlet port fluidically connected to said fluidic chamber;
   characterized therein that said fluidic chamber comprises at least one detachable and/or removable surface for the cultivation and/or analysis of surface dwelling multicellular layers. Surface-dwelling multicellular layers as referred to herein relate to biological monolayer or multilayer structures comprising one or more types of mammalian cells, insect cells, yeast cells, fungal cells, plant cells, microbial cells, bacterial cells, cellular vesicles, cellular organelles or tissue sections. In particular embodiments said multicellular layer composed of microbial cells is a biofilm.

More particularly, the flow of the fluid through said fluidic chamber is laminar, preferably having a Reynolds number smaller than 100. More particularly, the flow of the fluid through said fluidic chamber provides a linear flow profile over the entire width of the fluidic chamber, with the exception of the area (10%) adjacent to the side walls.

In a particular embodiment, the fluidic device according to the present invention provides in a device wherein said fluidic chamber comprises a bottom surface, two side surfaces and a top surface, wherein at least part of said top or bottom surface are detachable and/or removable. More particularly, at least part of said fluidic chamber is transparent for optical imaging, for microscopy, and/or for fluorescence imaging, thereby providing an imaging observation site. More particularly, the fluidic device further comprising observation zones and/or sensors located on or in one or more surface enclosing said fluidic chamber, for observing and/or monitoring the formation and/or cultivation of surface-dwelling multicellular layers on said detachable and/or removable surface. More particularly, said detachable and/or removable surface is made from or provided with an adherent surface material suitable for the adherence of the multicellular layer, wherein said surface material models a surface likely to be involved in cell adhesion and/or multicellular layer formation.

In a particular embodiment, the fluidic device according to the present invention provides that said surface-dwelling multicellular layer adherent surface material is chosen from the group comprising aluminum, stainless steel, silver, copper, hydroaxyapatite, silicon, latex, urethane, PVC, ceramic, steel, gold, titanium, polyethylene, polysiloxanes, biocompatible glasses, poly-methylmethacrylate, Teflon (or PTFE), polypropylene, polystyrene, polyamides, polyethers, polyesters, coated block polymers of polyethylene oxide (PEO), polypropylene oxide (PPO), polybutylene oxide (PBO), food film polymers polycarbonate filters and minerals.

In a further particular embodiment, the fluidic device according to the present invention provides that said fluidic device further comprising an inlet flow distributor for distributing incoming fluid over the fluidic chamber and optionally an outlet flow distributor for guiding the outflowing fluid from the fluidic chamber to the outlet. More particularly, said inlet flow distributor or part thereof slopes downward relative to the horizontal position of the fluidic chamber.

In a further particular embodiment, the fluidic device according to the present invention provides that the depth of the fluidic chamber ranges between 0.1 and 5 mm. More particularly, said fluidic device is made from a polymeric material and wherein said detachable and/or removable surface wall of said fluidic device is made from a material other than said polymeric material.

In a further embodiment, the present invention relates to a method for cultivating and monitoring surface-dwelling multicellular layers, comprising:

a) providing a fluidic device comprising:
a fluidic chamber comprising one or more surfaces enclosing the fluidic chamber through which fluid can flow along a flow path, said fluidic chamber comprising at least one detachable and/or removable surface for cultivating multicellular layers;
an inlet port fluidically connected to said fluidic chamber;
an outlet port fluidically connected to said fluidic chamber;

b) dispensing a flowing liquid growth medium, optionally comprising microorganisms, animal cells, plant cells or fungi cells, into said fluidic chamber, said growth medium flowing across said detachable and/or removable surface, thereby generating a surface-dwelling multicellular layer on said detachable and/or removable surface; and

c) monitoring the multicellular layer on said detachable and/or removable surface under varying conditions.

In particular embodiments said surface dwelling multicellular layer composed of unicellular organism cells (e.g. microbes) is a biofilm.

More particularly the method provides that said varying conditions comprise different types of fluid media, different fluid flow rates, different temperatures and/or combinations thereof.

In a further embodiment, the present invention relates to a flow distributor for distributing a fluidic flow from a narrow inlet channel to a wider fluidic channel, wherein said flow distributor comprises a first distribution region shaped as an isosceles trapezoid fluidically connected through the larger of the two parallel sides of said isosceles trapezoid to a second rectangular shaped distribution region, wherein the narrow inlet channel is fluidically connected to the smaller of the two parallel sides of said first distribution region and said wider fluidic channel is fluidically connected to the second distribution region, characterized in that said second distribution region slopes downward relative to the horizontal position of said wider fluidic channel.

### BRIEF DESCRIPTION OF THE DRAWING

The following description of a specific embodiment of the invention is merely exemplary in nature and is not intended to limit the present teachings, their application or uses.

FIG. 1 illustrates a specific embodiment of the fluidic device according to the present invention.

FIG. 2 illustrates fluidic simulations (Fluent) in a fluidic device according to an embodiment of the present invention

FIG. 3 illustrates a specific embodiment of the fluidic device according to the present invention.

FIG. 4 is a graphical representation of the mean adhesion values of ATCC 12228 and ATCC 35984 on PDMS surfaces either bare, coated with TSB or with serum expressed as the log 10 of adherent bacteria/mm².

FIG. 5 is a plot showing the surface nitrogen abundance on PDMS surfaces with respect to the quantity of serum contained in the solutions used to coat them.

FIG. 6 is graphical representation of the nitrogen abundance on the surface of PDMS coated with either diluted serum, pure albumin at 1 g/L, albumin and fibrinogen both at 0.5 g/L, or TSB.

FIG. 7 illustrates a specific embodiment of the fluidic device according to the present invention.

### DETAILED DESCRIPTION

The present invention will be described with respect to particular embodiments but the invention is not limited thereto.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” when referring to recited members, elements or method steps also include embodiments, which “consist of” said recited members, elements or method steps.

Furthermore, the terms first, second, third and the like in the description, are used for distinguishing between similar elements and not necessarily for describing a sequential or chronological order, unless specified. It is to be understood that the terms so used are interchangeable under appropriate circumstances and that the embodiments described herein are capable of operation in other sequences than described or illustrated herein.

All documents cited in the present specification are hereby incorporated by reference in their entirety.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art.

The present invention provides in a fluidic device providing in the growth of a surface-dwelling multicellular layer under controlled dynamic conditions. The device allows the cultivation and growth of a multicellular layer on a surface adherent material, but also offers the possibility to monitor and analyze the multicellular layer under changing and controlled conditions. The contact interface on which the multicellular layer is grown is removable and/or detachable from the fluidic chamber, thereby being adaptable and allowing alteration. Also the control of the fluid flow is highly regulated throughout the fluid cell and the device offers a high flexibility in analysis possibilities. In addition, all system parts of the fluidic device are autoclavable, a property of major importance with regard to biosafety and environmental issues. In particular embodiments said multicellular layer is a biofilm.

Surface-dwelling multicellular layers as referred to herein relate to biological monolayer or multilayer structures comprising one or more types of mammalian cells, insect cells, yeast cells, fungal cells, plant cells microbial cells, bacterial cells, cellular vesicles, cellular organelles or tissue sections. Typically, the cultivation and/or growth of surface-dwelling multicellular layers are known for the growth of bacterial, archaeal or yeast cells in the form of biofilms. However, also other cell type growth and adhesion may be assessed such as for instance in a particular embodiment the adhesion of animal cells upon a layer of a chosen compound (i.e. antibodies, receptors, extracellular matrix molecules, biomaterial . . . ). Also, in another particular embodiment fungi and/or plant cells could also be used in the devices according to the present invention.

Accordingly, useful cells include prokaryotes and eukaryotes such as mammalian cells including hybridoma cells, insect cells, yeast cells, fungal cells, plant cells, microbial cells, bacterial cells, tissue sections and/or protist cells comprising protozoa, algae and fungal cells. Mammalian cells may be derived from any recognized source with respect to species (e.g. human, rodent, simian), tissue source (brain, liver, lung, heart, kidney, skin, muscle) and cell type (e.g. epithelial, endothelial). In addition, cells which have been transfected with recombinant genes may also be cultured using the present invention.

Suitable cell lines may be comprised within e.g. the American Type Culture Collection and the German Collection of Microorganisms and Cell Cultures.

Non-limiting examples of useful mammalian cell lines include animal and human cell lines such as Chinese hamster ovary (CHO) cells, Chinese hamster lung (CHL) cells, baby hamster kidney (BHK) cells, COS cells, HeLa cells, THP cell lines, Jurkat cells, hybridoma cells, carcinoma cell lines, hepatocytes, primary fibroblasts, endothelial cells, tumor cell lines and the like. In particular embodiments tumor cell lines or carcinoma cell lines are envisaged.

Suitable insect cell lines include but are not limited to Lepidoptera cell lines such as Spodoptera frugiperda cells and Trichoplusia ni cells.

Non-limiting examples of fungal cells useful in the present invention include the phyla Ascomycota, Basidiomycota, Chytridiomycota, and Zygomycota as well as the Oomycota and all mitosporic fungi. Representative groups of Ascomycota include, e.g., Neurospora spp., Eupenicillium (or Penicillium) spp., Emericella (or Aspergillus) spp., Eurotium (or Aspergillus) spp., and the true yeasts listed above. Examples of Basidiomycota include mushrooms, rusts, and smuts. Representative groups of Chytridiomycota include, e.g., Allomyces spp., Blastocladiella spp., Coelomomyces spp., and aquatic fungi. Representative groups of Oomycota include, e.g. saprolegniomycetous aquatic fungi (water molds) such as Achlya spp. Examples of mitosporic fungi include Aspergillus spp., Penicillium spp., Candida spp. and Altemaria spp. Representative groups of Zygomycota include, e.g., Rhizopus spp. and Mucor spp.

Fungal cells may be yeast cells. Non-limiting examples of useful yeast cells include ascosporogenous yeast (Endomycetales), basidiosporogenous yeast, and yeast belonging to the Fungi Impeffecti or Deuteromycota (Blastomycetes). The ascosporogenous yeasts are divided into the families Spermophthoraceae and Saccharomycetaceae. The latter is comprised of four sub-families, Schizosaccharomycoideae (e.g., genus Schizosaccharomyces including S. pombe), Nadsonioideae, Lipomycoideae, and Saccharomycoideae (e.g., genera Pichia including P. pastoris, P. guillermondii and P. methanolio), Kluyveromyces including K. lactis, K. fragilis and Saccharomyces including S. carlsbergensis, S. cerevisiae, S. diastaticus, S. douglasii, S. klayveri, S. norbensis or S. oviformis). The basidiosporogenous yeasts include the genera Leucosporidim, Rhodosporidium, Sporidiobolus, Filobasidium, and Filobasidiella. Yeasts belonging to the Fungi Impeffecti are divided into two families, Sporobolomycetaceae (e.g., genera Sporobolomyces and Bullera) and Cryptococcaceae (e.g., genus Candida including C. maltose). Other useful yeast host cells are Hansehula polymorpha, Yarrowia lipolytica, and Ustilgo maylis.

Fungal cells may be filamentous fungal cells including all filamentous forms of the subdivision Eumycota and Oomycota. Filamentous fungi are characterized by a vegetative mycelium composed of chitin, cellulose, glucan, chitosan, mannan, and other complex polysaccharides. Vegetative growth is by hyphal elongation and carbon catabolism is obligatory aerobic. In contrast, vegetative growth by yeasts such as Saccharomyces cerevisiae is by budding of a unicellular thallus and carbon catabolism may be fermentative. In a more preferred embodiment, the filamentous fungal host cell is a cell of a species of, but not limited to the genera, Acremonium, Aspergillus, Fusarium, Humicola, Mucor, MyceliopHthora, Neurospora, Penicillium, Thielavia, Tolypocladium, and Trichoderma or a teleomorph or synonym thereof.

Suitable plant cells for use in the present invention include dicotyledonous plant cells, examples of which are Arabidopsis thaliana, tobacco, potato, tomato, and leguminous (e.g. bean, pea, soy, alfalfa) cells. It is, however, contemplated that monocotyledonous plant cells, e.g. monocotyledonous cereal plant cells such as for example rice, rye, barley and wheat, may be equally suitable.

A multicellular layer of adherent microorganisms is often referred to as a biofilm. The microorganisms in the biofilm may include a mixture of bacteria, for example, including Gram-positive, Gram-negative bacteria, anaerobic bacteria, aerobic bacteria, and any combination thereof. The Gram-positive bacteria includes without limitation to Enterococcus faecalis, Staphylococcus aureus, Staphylococcus epidermidis, Streptococcus viridans, Listeria monocytogenes, Bacillus cereus or any combination thereof. The Gram-negative bacteria includes without limitation to Escherichia coli, Klebsiella pneumoniae, Proteus mirabilis, Pseudomonas aeruginosa and any combination thereof. Bacteria may be aerobic or anaerobic, the anaerobic bacteria is Streptococcus anginosus, Streptococcus australis, Streptococcus constellatus, Streptococcus mitis, Enterobacter spp., Actinomyces spp., Veillonella spp., Prevotella melaninogenica, Fusobacterium periodonticum or any combination thereof. The aerobic bacteria includes without limitation to strains of the following genera Aeromonas, Bacillus, Burkholderia, Flavobacterium, Listeria, Microbacterium, Pseudomonas, Salmonella, Staphylococcus or any combination thereof. In certain embodiments, the biofilm includes oral bacteria. The oral bacteria includes without limitation to Actinomyces viscosus, Actinomyces naselundii, Streptococcus mutans, Streptococcus sanguis, Streptococcus sobrinus, Lactobacillus casei, Lactobacillus acidophilius, Candida albicans, Actinobacillus actinomycetemcomitans, Veillonella parvula, Fusobacterium nucleatum subsp. polymorphum, Porphyromonas gingivalis, Neisseria spp., and any combination thereof.

In particular embodiments said multicellular layers as referred to herein comprise a combination of two or more different types of organisms such as for instance the combination of bacteria with yeast or the combination of human cell lines with bacteria.

The removable and/or detachable contact interface in the fluidic chamber provides that the contact surface can be removed from the fluidic chamber without the necessity to break parts of it or without having to apply forces that could disrupt the fore grown multicellular layer.

The devices and methods according to the present invention accordingly allow the cultivation of surface-dwelling multicellular layers under controlled dynamic flow conditions where these parameters are controlled over the entire scan observation area, thereby assuring a laminar flow parabolic velocity profile and constant shear forces over this area. The contact interface on which the multicellular layer is grown is removable and/or detachable from the fluidic chamber and is pressed against the fluidic chamber without the requirement of any other seals. Accordingly, only the contact interface on which the multicellular layer is grown is contaminated by the culture medium. The removable character of this contact interface also allows fast and easy adaptation depending on the needs and the type of multicellular layer to be analyzed. A surface of hydroxyapatite may for instance be used for experimenting with dental biofilms, a stainless steel plate may be used to simulate behavior in industry applications and silicone can be used to mimic the attachment of bacteria (biofilms) on biomaterials. When using observations zones for monitoring the multicellular layer, the observation may occur in real time depending on the properties of the surface to be used. Also, once the experiments are performed, the biomass of the surface-dwelling multicellular layer can easily be recovered as the surface can be removed and subjected to further surface analysis. Also, the temperature in the growth chamber is kept constant through good heat diffusion. If accurately calibrated, a simple equipment such as a Peltier or a hot plate is sufficient to maintain an optimized temperature control. The medium passing through the flow chamber is heated at the desired temperature.

In a first aspect, the present invention relates to a fluidic device the analysis of a surface, the fluidic device comprising:
a fluidic chamber comprising one or more surfaces enclosing the fluidic chamber through which fluid can flow along a flow path, wherein said fluidic chamber is characterized by having a Young's Modulus [E] ranging between 500 kPa and 5 MPa;
an inlet port fluidically connected to said fluidic chamber through an inlet flow distributor for distributing the incoming fluidic flow from said narrow inlet port to said wider fluidic chamber;
an outlet port fluidically connected to said fluidic chamber optionally through an outlet flow distributor for distributing the outflowing fluidic flow from said wider fluidic chamber to said narrow outlet port;

characterized therein that said fluidic chamber comprises at least one detachable and/or removable surface for analysis purposes.

In particularly, while several of the embodiments described herein provide in fluidic devices that are directed to the cultivation and/or analysis of surface-dwelling multicellular layers on the detachable and/or removable surface, it can be recognized that the devices according to the present invention can be used for various alternative analyses. The devices according to the present invention may for instance be used to test the anti-fouling properties of a surface, the resistance of a surface to the attachment of bacteria, but also to test the effects of certain fluids (being circulated through the fluidic device) onto a coated or uncoated surface. Accordingly, apart from creating a biofilm onto the removable and/or detachable surface, the same surface can be coated with a variety of chemical or biochemical structures which are further analyzed under a variety of circumstances to which the surface is submitted in the fluidic device according to the present invention. For instance the effect of certain chemical solutions such as detergents on a coated or uncoated surface can be assessed using the fluidic device according to the present invention.

In a particular embodiment, the present invention relates to a fluidic device particularly for the cultivation and/or analysis of surface-dwelling multicellular layers comprising:
a fluidic chamber comprising one or more surfaces enclosing the fluidic chamber through which fluid can flow along a flow path;
an inlet port fluidically connected to said fluidic chamber;
an outlet port fluidically connected to said fluidic chamber;
   characterized therein that said fluidic chamber comprises at least one detachable and/or removable surface for the cultivation and/or analysis of surface-dwelling multicellular layers.

In a particular embodiment the fluidic device according to the present invention comprises:
a fluidic chamber comprising one or more surfaces enclosing the fluidic chamber through which fluid can flow along a flow path, wherein said fluidic chamber is characterized by having a Young's Modulus [E] ranging between 500 kPa and 5 MPa, preferably ranging between 500 kPa and 2 MPa, more preferably ranging between 500 kPa and 1 MPa;
an inlet port fluidically connected to said fluidic chamber through an inlet flow distributor for distributing the incoming fluidic flow from said narrow inlet port to said wider fluidic chamber;
an outlet port fluidically connected to said fluidic chamber optionally through an outlet flow distributor for distributing the outflowing fluidic flow from said wider fluidic chamber to said narrow outlet port;
   characterized therein that said fluidic chamber comprises at least one detachable and/or removable surface in particularly for the cultivation and/or analysis of surface-dwelling multicellular layers.

In particular embodiments said multicellular layer composed of microbial cells is a biofilm.

Compared to other flow cell systems for growing multicellular layers such as biofilms in dynamic conditions, the fluidic device according to the present invention is characterized by having at least one interchangeable contact surface for the growth of the multicellular layer. Indeed the surface, on which the multicellular layer is cultivated, is removable and/or detachable from the fluidic device. This allows the user to change the cultivation surface depending on the type of surfaces one wants to study the multicellular layer formation on. Accordingly, the surface-dwelling multicellular layer formation is not limited to the type of material the fluidic chamber is made of. As one of the surfaces is detachable, the material from which this surface is made can be chosen by the user. Also, the detachable and/or removable surface for the cultivation and/or analysis of multicellular layers does not compromise the type of analysis options using for instance microscopy, spectroscopy or the recovery of biomass.

Also, the fluidic device according to the present invention offers the possibility to grow surface-dwelling multicellular layers in dynamic conditions under controlled conditions of the fluidic flux inside the fluidic chamber, thereby obtain the desired accuracy and reproducibility.

Alternatively, the detachable and/or removable surface is treated and/or coated with functional chemical or biochemical substances including, but not limited to proteins, peptides, glycoproteins (e.g. mucins), antibodies, DNA, polysaccharides, lipids or other biomolecules, nanostructures, resins, biocides, metals, metal ions, metal oxides or other analytes.

Alternatively, the devices according to the present invention may also be used to test the anti-fouling properties of a surface. Here fouling on the detachable and/or removable surface can be assessed. As the formation of fouling on a surface is correlated with biofilm formation on that surface, both uses of the devices according to the present invention are closely related.

As used herein, the term “fluidic chamber” refers to an enclosure that defines an interior space or cavity which can contain a fluid and through which a fluid can flow. The fluid is referred to as a substance that has the ability to flow and to conform to the boundaries of a container in which the substance is placed. Fluids include liquid and gas substances, and preferably liquid substances. Fluids include, but are not limited to, water, buffer, blood, preparative fluids, culture medium, reagents, organic solutions, inorganic solutions, and any fluid that can be used to conduct an assay.

The fluid in the fluidic device according to the present invention flows through the fluidic chamber according to a flow path (the path of flow of a fluid). A flow path can be the path of flow of a fluid through a flow chamber. For example, a flow path can be the path of flow of a fluid from an inlet port, through the interior of a chamber and to an outlet port. As used herein “inlet port” refers to a port through which a substance, e.g., a fluid, is introduced into a chamber, whereas “outlet port” refers to a port through which a substance, e.g., a fluid, is removed from a chamber. As used herein “fluidically connected” indicates that the items referred to are in fluid communication with each other.

In a particular embodiment, the fluidic device according to the present invention provides that the flow of the fluid through said fluidic chamber is laminar. More particularly, the Reynolds number of the flow of the fluid through said fluidic chamber is smaller than 100.

As used herein, the term “laminar” or “laminar flow”, with reference to a fluid, refers to the fluid flow being substantially non-turbulent. The degree of laminar flow can be characterized by the Reynolds number, which is a measurement of the tendency for turbulence to occur as described by the equation:

$$Re = \frac{ρ \cdot v \cdot L}{μ}$$

wherein Re is a Reynolds number, v is the average linear flow rate, L is a unit of length, p is the density of the fluid and μ is dynamic viscosity of the fluid (μ/ρ being the kinematic viscosity of the fluid). Turbulence in fluid flow occurs in flows characterized by high Reynolds (Re) numbers. Thus, laminar flow generally can be characterized by a Re less than about 100. In particular embodiments, laminar flow can be characterized by a Re less than about 90, or less than about 80, or less than about 70, or less than about 60, or less than about 50, or less than about 40, or less than about 30, or less than about 20 or less than about 10. Laminar flow can also be referred to as a streamlined, uniform flow of fluid near a solid boundary without any significant turbulence. A fluid path that has little to no bending, little to no sharp turns, little to no steep slopes, little to no recirculation paths, little to no retroflow, little to no eddy currents, and little to no whorls generally can provide for substantially laminar flow. The term is also used to describe a linear flow of fluid that has a controllable linear velocity at the surface of a flow chamber provided and that is non-disruptive to an element (e.g., a living cell or an analyte) of a process, e.g., a biological or biochemical process, in the flow chamber.

As used herein, substantially laminar flow inside the fluidic chamber, with reference to a fluid, is a flow of fluid that is at least about 80% laminar, at least about 85% laminar, at least about 90% laminar, at least about 95% laminar, at least about 96% laminar, at least about 97% laminar, at least about 98% laminar, at least about 99% laminar, or at least about 100% laminar. The laminar flow in the fluidic chamber avoids the occurrence of dead zones in the chamber. Accordingly, any non attached cells of the multicellular layer or unattached coating residues are cleared from the fluidic chamber through the constant laminar flow prohibiting them from interfering with the analysis.

For instance, for the flow of water at 30° C. in the fluidic chamber of the device according to the present invention (with for instance a characteristic length of 0.001 m and a fluid velocity of 0.001 m/s) the calculated Reynolds Number is about 1.3. Since the speed of the liquid has a plain linear contribution to Re (Reynolds Number). In particular embodiments, the typical fluid flow velocities in the devices according to the present invention do not exceed 0.01 m/s for multicellular layer growth to occur and the density of medium used for the multicellular layer growth about 1000 kg/m³.

In a particular embodiment, the fluidic device according to the present invention provides that the flow in the fluidic chamber has a Hydraulic Retention Time (HRT=Volume [m³]/Flow [m³/s]) ranging between 5 seconds and 30 minutes, more particularly, between 10 seconds and 15 minutes, more particularly, between 15 seconds and 5 minutes, more particularly, between 20 seconds and 60 seconds and more particularly, about 30 seconds. Typical fluid flow rates through the fluidic device range between 0.05 ml/min and 10 ml/min.

In a particular embodiment, the fluidic device according to the present invention provides that the flow of the fluid through said fluidic chamber provides a linear flow profile over the entire width of the fluidic chamber, with the exception of the area 10% from the side walls. More particularly, the present invention provides that the flow of the fluid through said fluidic chamber provides a linear flow profile over the entire width of the fluidic chamber, with the exception of the area at a distance from the side walls which is smaller than 10%, 9%, 8%, 7.5%, 7%, 6.5%, 6.25% or 6% of the total width of the fluidic chamber. Accordingly, the present invention provides that the flow of the fluid through said fluidic chamber provides a linear flow profile over at least 80% of the total width of the fluidic chamber, more particularly, a linear or laminar flow profile is provided over at least 80%, 82%, 84%, 85%, 86%, 87%, 87.5% or 88% of the total width of the fluidic chamber. Accordingly, the fluid passes through the fluidic chamber in a laminar manner thereby generating a fluid flow which provides an equal velocity over the entire width of the fluidic chamber, the only deviation from this occurring in the area near the side walls. Typically the area where the deviation occurs is located in the area from the side walls which is smaller than 10%, 9%, 8%, 70.5%, 7%, 6.5%, 6.25% or 6% of the total width of the fluidic chamber. For instance, when the chamber is 15 mm wide, the area where the deviation occurs is in the 1.5 mm area or less from the side walls.

In a particular embodiment, the fluidic device according to the present invention provides that said fluidic chamber comprises a bottom surface, two side surfaces and a top surface, wherein at least part of said top or bottom surface are detachable and/or removable. More particularly, the detachable and/or removable surface is the bottom surface.

In a particular embodiment, the fluidic device according to the present invention provides that at least part of said fluidic chamber is transparent for optical imaging, for microscopy, and/or for fluorescence imaging, thereby providing an imaging observation site. For instance at least part of the fluidic chamber is made transparent for observation and/or monitoring the removable and/or detachable surface, in particularly the surface-dwelling multicellular layer, through classical light microscopy. The fluidic chamber typically provides a sufficient optical transparency and clarity to permit observation of the removable and/or detachable surface, in particular the multicellular layer and the cells thereon. The transparent characteristic of part of the fluidic chamber is especially useful for real time imaging of surface, in particular the multicellular layer.

In a particular embodiment the imaging of the removable and/or detachable surface, in particularly the surface-dwelling multicellular layer, occurs using a confocal microscope thereby permitting 3D images to be made.

More particularly, at least part of the fluidic chamber is casted in a transparent or translucid, heat-resistant, polymeric material which Young's Modulus [E] is comprised between 500 kPa and 5 MPa, preferably ranging between 500 kPa and 2 MPa, more preferably ranging between 500 kPa and 1 MPa. The rigidity is an important parameter for the device according to the present invention as the removable surface of the fluidic chamber is pressed against the rest of the device to ensure complete sealing of the fluidic chamber. This makes materials with a too low Young's modulus unsuitable because the open flow chamber inside the material would lose its shape under pressure applied to seal the device. Accordingly, materials such as PDMS (Poly-dimethylsiloxane) (good rigidity, transparency and heat-resistance up to 300° C.) are used. Typically the PDMS is prepared with a ratio between base and cross-linking agent ranging between 1:10 and 1:30, preferably 1:20, and baking the polymers between 80° C. and 110° C., preferably about 95° C., for 30 minutes to 2 hours, preferably about 1 hour.

Alternatively to PDMS, also other materials such as acrylate based polymers, fluoroelastomers and styrenic polymers characterized by having a Young's Modulus [E] comprised between 500 kPa and 5 MPa, preferably ranging between 500 kPa and 2 MPa, more preferably ranging between 500 kPa and 1 MPa.

The plates ensuring the pressure that maintains the removable surface and the fluidic chamber together can be made of any hard (Young modulus superior to 10 GPa), heat resistant material such as steel, aluminum or plastic polymer. The pressure can be provided through any commonly known method in the art.

In a particular embodiment, the fluidic device according to the present invention further comprises observation zones and/or sensors located on or in one or more surface enclosing said fluidic chamber, for observing and/or monitoring the removable and/or detachable surface, in particularly the formation and/or cultivation of the surface-dwelling multicellular layer on said detachable and/or removable surface.

As referred herein the “observation zones” and/or “sensors” are defined features in the walls of the fluidic chamber that allow observation using commonly known detection methods, such as for instance observation through (real-time) light microscopy. More particularly, one of the walls of the fluidic chamber is provided with an array of “observation fields” displaying the same set of conditions. Pictures taken from various observation fields can be statistically treated as repetitions of the same experiment. Observation fields positioned along different flow path lines can be considered independent repetitions of the same experiment.

In a particular embodiment of the present invention the sensors are crafted on the surface, said sensors being chosen from, but not limited to, for example, microelectrodes measuring gas concentration, pH, nutrient levels and redox potential of the local environment.

Another advantage of the removable and/or detachable surface is that upon finalization of the experiment, the surface on which in particular embodiments the multicellular layer has grown may be detached and recovered, preferably sterilely, for further analysis. Accordingly, of the detached surface properties such as surface composition, biomass recovery, and mechanical properties assessment (rheometry) can be assessed using adequate methods and techniques such as XPS or mass spectrometry (for analysis of the surface composition), Rheometer electron microscopy (for assessment the mechanical properties). The detached surface may also be treated in various ways to assess the resistance of multicellular layers towards a certain set of conditions, an aspect which is useful when the treatment conditions are unsuitable for passing through the fluidic device.

In a particular embodiment, the fluidic device according to the present invention provides that said detachable and/or removable surface is made from or provided with a surface-dwelling multicellular layer adherent surface material, wherein said surface material models a surface likely to be involved in cell adhesion and/or formation of a surface-dwelling multicellular layer. As multicellular layer formation on a surface is closely linked to the formation of fouling, the devices according to the present invention also envisage the analysis of the anti-fouling properties of a surface. Therefore fouling on the detachable and/or removable surface can be assessed by using a surface material likely to reject and withhold cell adhesion and/or multicellular layer formation (e.g. Pluronics coatings (brush polymers)).

In a particular embodiment, the fluidic device according to the present invention provides that said adherent surface material is chosen from the group comprising aluminum, stainless steel, silver, copper, hydroaxyapatite, silicon, latex, urethane, PVC, and ceramic, steel, gold, titanium, polyethylene, polysiloxanes, biocompatible glasses, poly-methylmethacrylate (PMMA-contact lenses material), Teflon (or PTFE), polypropylene, polystyrene, polyamides, polyethers, polyesters, coated block polymers of polyethylene oxide (PEO), polypropylene oxide (PPO), polybutylene oxide (PBO) known to be pluronics used as anti-adhesive coatings, hydrogels, food film polymers, polycarbonate filters (to study the effect of the diffusion of a molecule on the multicellular layer coming from an underlying solution or agar medium) or minerals (to study biofilm formation on river rocks).

Alternatively, the fluidic device according to the present invention provides that said detachable and/or removable surface is provided with an adherent surface material comprising proteins, glycoproteins (e.g. mucins) antibodies, DNA, polysaccharides, lipids or other biomolecules for the purpose of adhering cells and/or other components.

The type of surface material is typically dependant on the type of sterilization that is required. For sterilization through autoclaving, envisaged surfaces are surfaces resistant to humid heat (121° C. for at least 20 minutes) such as for instance glass surfaces topped with all sorts of coatings such as for instance metal deposited by CVD (chemical vapor deposition), polymers layered thereupon, sprayed ions, mineral layers (hydroxyapatite) or other bulk materials such as steel, alloys, metals, mineral rocky materials and polymers so long as their mechanical rigidity is sufficient (Young modulus [E] above 10 GPa).

For other sterilization methods (e.g. Gamma rays, electron beams, UV, ethylene oxide, . . . ) only the mechanical rigidity (E>10 GPa) forms a structural limit. Alternatively, the surface material is backed up by a more resistant supporting material providing the structure with the required mechanical rigidity.

In a particular embodiment, the fluidic device according to the present invention further comprises an inlet flow distributor for distributing incoming fluid over the fluidic chamber and optionally an outlet flow distributor for guiding the outflowing fluid from the fluidic chamber to the outlet. More particularly, the flow distributor distributes the incoming fluid flow evenly over the fluidic chamber.

In particular the fluidic device according to the present invention comprises an inlet flow distributor which comprises:
a first distributor region shaped as an isosceles trapezoid;
a second rectangular shaped distributor region;
   wherein said first distributor region is fluidically connected to the narrow inlet channel through the smaller of the two parallel sides and fluidically connected to said second rectangular shaped distribution region through the larger of the two parallel sides of said isosceles trapezoid, wherein said second rectangular shaped distribution region is further fluidically connected to said fluidic chamber, characterized therein that said second distribution region slopes downward relative to the horizontal position of said fluidic channel.

More particularly, the inlet flow distributor or part thereof slopes downward relative to the horizontal position of the fluidic chamber. More particularly, the slope angle relative to the direction of the inlet port or the fluidic chamber ranges between 15° and 75°, more particularly between 30° and 60°, more particularly between 40° and 50° and more particularly, about 45°.

In a particular embodiment the slope angle relative to the direction of the inlet port or the fluidic chamber of the bottom surface is larger compared to the slope angle relative to the direction of the inlet port or the fluidic chamber of the top surface. More particularly, the slope angle relative to the direction of the inlet port or the fluidic chamber of the top surface ranges between 15° and 75°, more particularly between 30° and 60°, more particularly between 40° and 50° and more particularly, about 45°, whereas the slope angle relative to the direction of the inlet port or the fluidic chamber of the bottom surface is larger than 50°, 60°, 75°, 80°, 85° and more particularly, about 90°.

In a particular embodiment the slope angle relative to the direction of the inlet port or the fluidic chamber of the top surface is 45°, while the slope angle relative to the direction of the inlet port or the fluidic chamber of the bottom surface is 90°.

The angles of the top and bottom surfaces of the flow distributor are chosen to allow an even and efficiently spread of the liquid in the fluidic chamber, while maintaining a good hydrodynamic profile without the need to lengthen the device.

In a particular embodiment, the fluidic device according to the present invention provides that the depth of the fluidic chamber ranges between 0.1 and 5 mm.

In a particular embodiment, the fluidic device according to the present invention provides that said fluidic device is made from a polymeric material and wherein said detachable and/or removable surface wall of said fluidic device is made from a material other than said polymeric material. More particularly, said polymeric material is PDMS.

In a particular embodiment, the fluidic device according to the present invention is made from a single piece of material, preferably a polymeric material such as PDMS. As used herein, the wording “single piece” or “one piece” refers to a structure, in particularly a fluidic device, made from a single piece of material. In particularly such a single-piece or one-piece structure, in particularly a fluidic device, is a monolithic structure made entirely from the same type of material. More particularly, the different parts of the fluidic device according to the present invention, with the exception of the detachable surface, are completely integrated into the structure forming a single and undividable structure the parts of which are being permanently connected to each other. In particularly the fluidic device according to the present invention is manufactured or casted entirely as a whole.

The present invention further provides in a method for performing an analysis between a surface of interest and a fluid of interest, comprising:

a) providing a fluidic device comprising:
a fluidic chamber comprising one or more surfaces enclosing the fluidic chamber through which fluid can flow along a flow path, said fluidic chamber comprising at least one detachable and/or removable surface of interest;
an inlet port fluidically connected to said fluidic chamber;
an outlet port fluidically connected to said fluidic chamber;

b) dispensing fluid of interest through said fluidic device, thereby contacting the fluid of interest with the surface of interest; and

c) monitoring interaction between the surface of interest and a fluid of interest under varying conditions.

As indicated above, while several of the embodiments described herein provide in methods for the cultivation and/or analysis of surface-dwelling multicellular layers on the detachable and/or removable surface, it can be recognized that the methods according to the present invention can also be used for various alternative analyses. The methods according to the present invention may for instance be used to test the anti-fouling properties of a surface, the resistance of a surface to the attachment of bacteria, but also to test the effects of certain fluids (being circulated through the fluidic device) onto a coated or uncoated surface. Accordingly, apart from creating a biofilm onto the removable and/or detachable surface, the same surface can be coated with a variety of chemical or biochemical structures which are further analyzed under a variety of circumstances to which the surface is submitted in the fluidic device according to the present invention. For instance the effect of certain chemical solutions such as detergents or cleaning solutions on a coated or uncoated surface can be assessed using the fluidic device according to the present invention.

More particularly, the method according to the present invention is a method for cultivating and monitoring surface-dwelling multicellular layers comprising:

a) providing a fluidic device comprising:
a fluidic chamber comprising one or more surfaces enclosing the fluidic chamber through which fluid can flow along a flow path, said fluidic chamber comprising at least one detachable and/or removable surface for the cultivating surface-dwelling multicellular layers;
an inlet port fluidically connected to said fluidic chamber;
an outlet port fluidically connected to said fluidic chamber;

b) dispensing a flowing liquid growth medium, optionally comprising microorganisms, animal cells, plant cells or fungi cells, into said fluidic chamber, said growth medium flowing across said detachable and/or removable surface, thereby generating a surface-dwelling multicellular layer on said detachable and/or removable surface; and

c) monitoring the surface-dwelling multicellular layer on said detachable and/or removable surface under varying conditions.

In particular embodiments said surface-dwelling multicellular layer composed of microbial cells is a biofilm.

More particularly, the method according to the present invention provides that said varying conditions comprise different types of fluid media, different fluid flow rates, different temperatures, different compounds and/or combinations thereof. In particular, the analysis between a surface of interest and a fluid of interest and particularly the monitoring of the multicellular layer, may occur upon challenging surface of interest, in particularly comprising the multicellular layer, with a varying set of conditions. These conditions may be physical conditions such as the temperature or fluid flow rates, and/or chemical conditions where the surface of interest, in particularly comprising the multicellular layer, is submitted to different types of fluid media and/or compounds such as for instance antibiotics, viruses which may be introduced into the flow cell after or at the same time as the growth medium.

Using the device according to the present invention and associated methods for using these, an operator is able to tune several conditions in the device, which may for instance influence the formation and/or cultivation of a surface-dwelling multicellular layer. Several parameters can be varied in the devices according to the present invention including for instance:

a) the contact surface, either the bulk surface material or the coating thereon of the removable surface as already detailed above;
b) the type of fluid medium used to grow the surface-dwelling multicellular layers, which can be any type of liquid characterized by a moderate viscosity such as for instance water, liquid food (e.g. milk), human or animal fluids, laboratory media, wastewater, vegetal extracts, cell extracts, solvents, etc.;
c) the fluid flow typically ranging between 0.1 and 10 ml/min (HRT: ranging between 5 minutes and a few seconds);
d) the temperature for growth of the surface-dwelling multicellular layer, typically ranging between 0° C. and 99° C.;
e) the type of inoculums used, typically the type of suspension of cells of any concentration injected for a chosen period of time. Also a co-injection of inoculum and medium and/or buffer solution may be envisaged;
f) the sequence of injection of various inoculi and media in different orders;
g) either a unidirectional mode where fresh medium is injected and waste is discarded or a (partial) circular mode where a fixed quantity of medium is circulating during the time of the experiment and is not discarded in a waste bottle may be envisaged.

The cleaning of the device according to the present invention is also an important aspect as cleaning methods such as autoclaving kill all living organisms dwelling in the device. After autoclaving all the parts of the system are removed and washed with mild detergent and distilled water, optionally an ultrasonic treatment followed by extensive cleaning with distilled water can be used to remove any remaining adherent particles in tubes and on fluidic device. This cleaning method is fast and severe and is the safest regarding biosafety issues. Cleaning allows the device to be reused with a new removable surface after cleaning. The device according to the present invention allows such severe cleaning methods. While other, less efficient cleaning methods may be used as well (e.g. using sodium hypochlorite to kill bacteria and sterilize the system, UV or ethanol treatment), these methods are considered less severe compared to autoclaving. Moreover, such less efficient methods may lead to extensive use of potentially harmful oxidizing chlorinated substances which can be avoided with autoclaving.

In yet another particular embodiment the present invention also relates to a fluidic device according to the invention, wherein said device comprises at least two fluidic chambers with corresponding inlet and outlet ports and distributors, wherein said two fluidic chambers are arranged adjacent to each other and are separated by a detachable and/or removable surface.

In particular, the two fluidic chambers are placed with the open side of their channel face to each other. In between these two, a detachable and/or removable surface is inserted. Preferably said detachable and/or removable surface is a membrane. Accordingly, the porosity and composition of the membrane can be chosen according to experimental needs. Membrane dimensions preferably exceed the flow channel dimensions by at least 2 mm to each side. The membrane dimensions preferably not exceed 60 mm×24 mm.

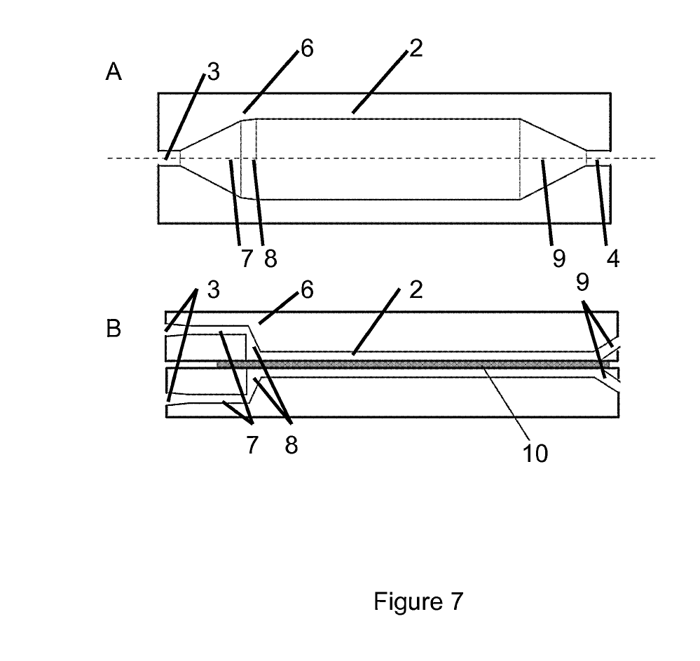

The three pieces (the two fluidic chambers and the detachable and/or removable surface) are pressed together between plates in order to ensure good sealing of the setup. The resulting device is composed of two inlets, two liquid distribution systems, two parallel channels and two outlets. The mounted system is represented in FIG. 7. Each flow chamber can be fed with separate liquids

The flow rate through both fluidic chambers can be identical such that the pressure on both sides of the membrane is identical. In such a set-up no pressure difference will be present over the membrane and the transfer of material from one side of the membrane to the other side will be the result of diffusion. Alternatively, the flow rate in the two fluidic chambers is non-identical thereby creating a pressure difference over the membrane thereby providing a forced transfer of materials from one fluidic chamber to the other. The latter being particularly used for mimicking the flow surrounding membranes used in industrial processes.

Accordingly, in a further embodiment the present invention provides in a fluidic device comprising:
a first fluidic arrangement comprising
   a first fluidic chamber comprising one or more surfaces enclosing the fluidic chamber through which fluid can flow along a flow path, wherein said first fluidic chamber is characterized by having a Young's Modulus [E] ranging between 500 kPa and 5 MPa, preferably ranging between 500 kPa and 2 MPa, more preferably ranging between 500 kPa and 1 MPa;
   a first inlet port fluidically connected to said first fluidic chamber through a first inlet flow distributor for distributing the incoming fluidic flow from said narrow first inlet port to said wider first fluidic chamber;
   a first outlet port fluidically connected to said first fluidic chamber optionally through a first outlet flow distributor for distributing the outflowing fluidic flow from said wider first fluidic chamber to said narrow first outlet port; and;
a second fluidic arrangement comprising
   a second fluidic chamber comprising one or more surfaces enclosing the fluidic chamber through which fluid can flow along a flow path, wherein said second fluidic chamber is characterized by having a Young's Modulus [E] ranging between 500 kPa and 5 MPa, preferably ranging between 500 kPa and 2 MPa, more preferably ranging between 500 kPa and 1 MPa;
   a second inlet port fluidically connected to said second fluidic chamber through a second inlet flow distributor for distributing the incoming fluidic flow from said narrow second inlet port to said wider second fluidic chamber;
   a second outlet port fluidically connected to said second fluidic chamber optionally through a second outlet flow distributor for distributing the outflowing fluidic flow from said wider second fluidic chamber to said narrow second outlet port;

characterized therein that said first and second fluidic chamber comprise a common detachable and/or removable surface. Preferably said particular common detachable and/or removable surface is a membrane.

In yet another embodiment the present invention also relates to a flow distributor for distributing a fluidic flow from a narrow inlet channel to a wider fluidic channel, wherein said flow distributor comprises a first distribution region shaped as an isosceles trapezoid fluidically connected through the larger of the two parallel sides of said isosceles trapezoid to a second rectangular shaped distribution region, wherein the narrow inlet channel is fluidically connected to the smaller of the two parallel sides of said first distribution region and said wider fluidic channel is fluidically connected to the second distribution region, characterized in that said second distribution region slopes downward relative to the horizontal position of said wider fluidic channel.

More particularly, the second distribution region slopes downward relative to the horizontal position of the first distribution region. More particularly, the slope angle relative to the direction of the first distribution region ranges between 15° and 75°, more particularly between 30° and 60°, more particularly between 40° and 50° and more particularly, about 45°.

In a particular embodiment the slope angle relative to the direction of the first distribution region of the bottom surface of the second distribution region is larger compared to the slope angle relative to the direction of the first distribution region of the top surface of the second distribution region. More particularly, the slope angle relative to the direction of the first distribution region of the top surface of the second distribution region ranges between 15° and 75°, more particularly between 30° and 60°, more particularly between 40° and 50° and more particularly, about 45°, whereas the slope angle relative to the direction of the first distribution region of the bottom surface of the second distribution region is larger than 50°, 60°, 75°, 80°, 85° and more particularly, about 90°.

In a particular embodiment the slope angle relative to the direction of the first distribution region of the top surface of the second distribution region is 45°, while the slope angle relative to the direction of the first distribution region of the bottom surface of the second distribution region is 90°.

The angles of the top and bottom surfaces of the of the second distribution region are chosen to allow an even and efficiently spread of the liquid, while maintaining a good hydrodynamic profile.

### EXAMPLES

### Example 1

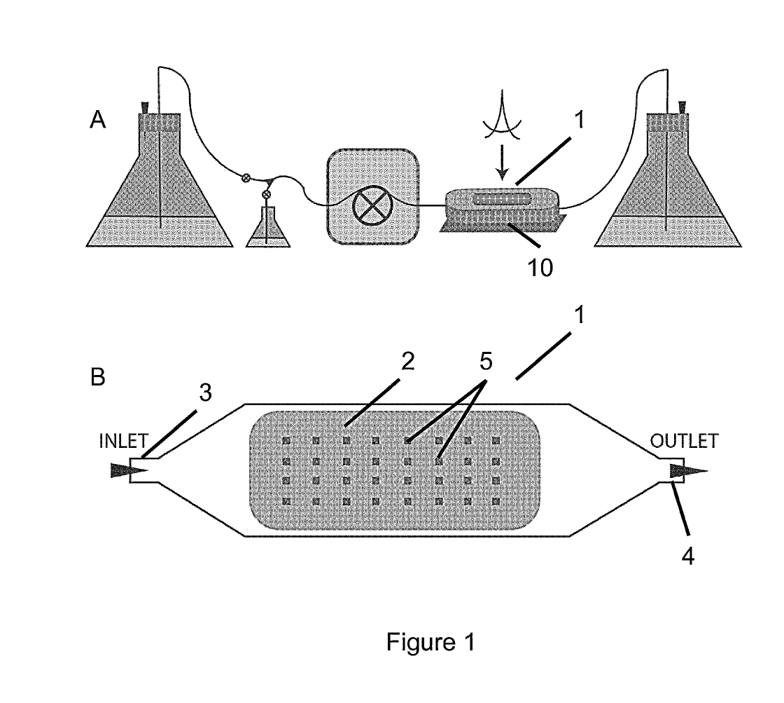

FIG. 1 illustrates a specific embodiment of the fluidic device according to the present invention. The fluidic device (1) is incorporated into a fluid flow system (FIG. 1A) where fresh medium is flown through the fluidic device using a pumping system. The fluidic device (1) comprises a fluidic chamber (2), an inlet (3) and an outlet (4). One of the surfaces of the fluidic chamber is removable and on this removable surface (10) formation of the surface-dwelling multicellular layer is monitored through an array of observation zones (5) as shown in FIG. 1B.

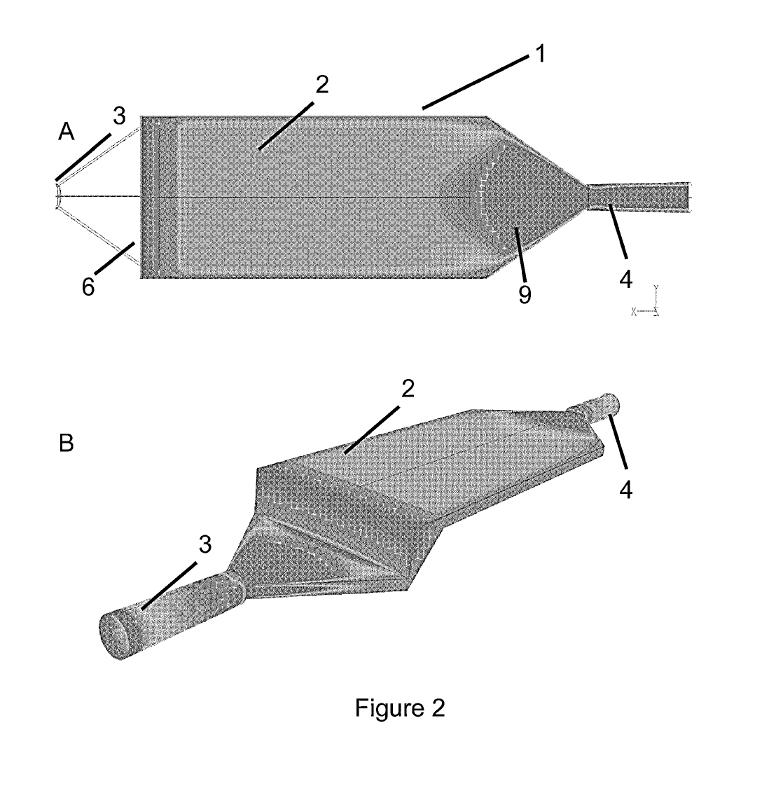

FIG. 2 shows fluidic simulations (Fluent) in a fluidic device (1) according to an embodiment of the present invention comprising a fluidic chamber (2), an inlet (3) and an outlet (4). The fluidic device is also provided with an inlet flow distributor (6) and an outlet flow distributor (9). The simulations show that the fluid flow is spread evenly in the fluidic chamber so that there is no dead zone in the chamber. The numerical analysis also shows that both speed profile and shear forces are constant on the entirety of the zone where the analysis of the multicellular layer occurs (a 25×15 mm zone in the center of the fluidic chamber). This ensures a fine-tuned control over experimental conditions in order to yield reproducible formation of the multicellular layer. In addition, the whole analysis area can be divided into a number of observation spots (as shown in FIG. 1B) considered to be repetitions from one another which makes the system interesting from the statistical point of view.

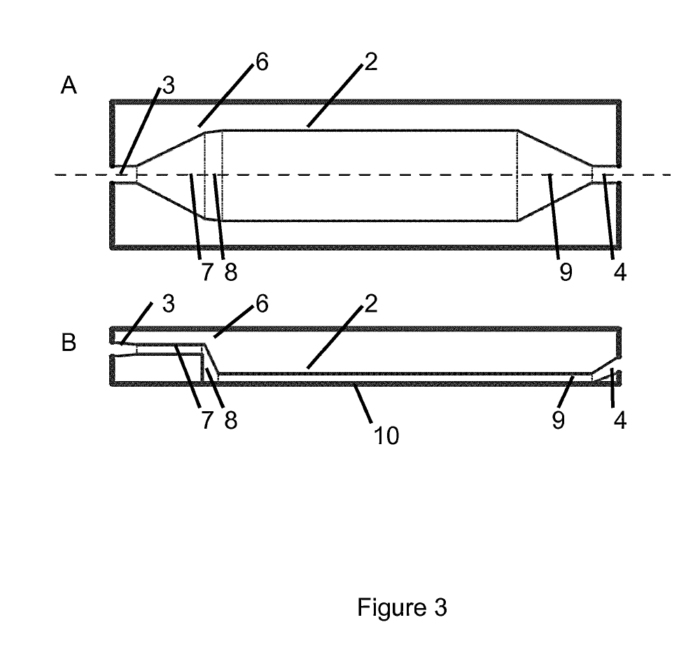

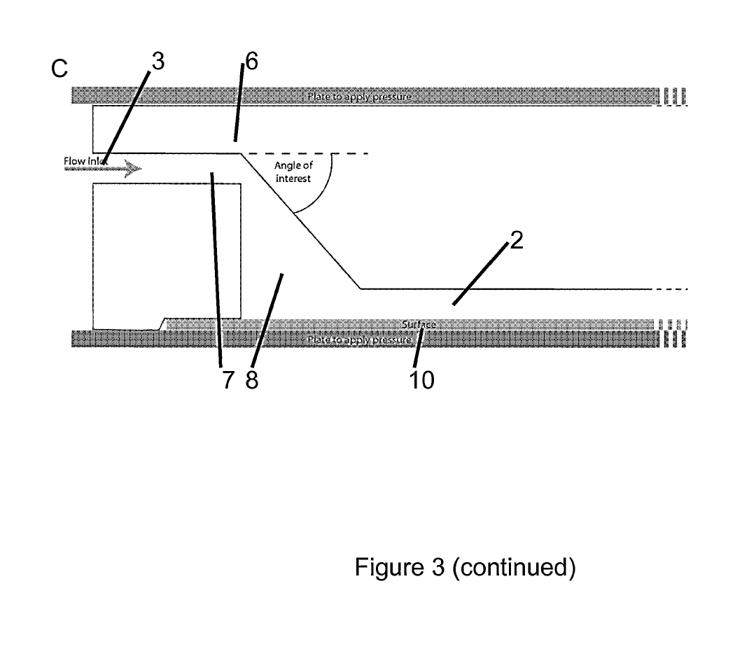

FIGS. 3A, 3B and 3C illustrate a specific embodiment of the fluidic device according to the present invention. The fluidic device (1) comprises a fluidic chamber (2), an inlet (3) and an outlet (4). The fluidic device is also provided with an inlet flow distributor (6) and an outlet flow distributor (9). One of the surfaces of the fluidic chamber is removable and on this removable surface (10) formation of the surface-dwelling multicellular layer is monitored. The inlet flow distributor (6) comprises a first distribution region (7) shaped as an isosceles trapezoid fluidically connected through the larger of the two parallel sides of said isosceles trapezoid to a second rectangular shaped distribution region (8), wherein the narrow inlet channel is fluidically connected to the smaller of the two parallel sides of said first distribution region and said wider fluidic channel is fluidically connected to the second distribution region, characterized in that said second distribution region slopes downward relative to the horizontal position of said wider fluidic channel. More particularly, the slope angle relative to the direction of the first distribution region of the top surface of the second distribution region is 45°, while the slope angle relative to the direction of the first distribution region of the bottom surface of the second distribution region is 90°.

### Example 2

In the present example, the fluidic device according to the present invention is used to study the adhesive behaviour of a bacterial species on a PolyDiMethylSiloxane (PDMS) surface (uncoated versus previously coated with proteins).

### A. Buffers and Media

Trypicase Soy Broth (TSB, Biorad) is prepared by adding 30 g of powder to 1 liter of distilled water. Phosphate buffer saline (PBS, Sigma) is obtained by dissolving a tablet in 200 ml of DI water, final pH 7.4. The solutions are autoclaved for sterilization.

### B. Bacterial Strains

The strains used in the present example are S. epidermidis ATCC 12228 and ATCC 35984. Stocks are streaked on agar plates containing TSB and incubated at 37° C. for 16 hours. All cultures are carried out in 50 ml of TSB in 250 erlenmeyer shaker flask, overnight, 37° C. and under 120 RPM agitation, from the inoculum of the agar plates. After filtration (5 μm porosity membrane) and centrifugation (5500 g for 15′ at 4° C.) of the cultures, the supernatant is discarded and the pellet is resuspended in 20 ml of PBS. The OD at 600 nm of the suspension is adjusted to a value of 2 by adding adequate volume of PBS.

### C. Conditioning Solutions

Two coating solutions are used: TSB and diluted adult bovine serum. Diluted bovine serum is obtained by diluting adult bovine serum (Sigma) ten-fold in PBS.

### D. Fluidic Device

The fluidic device according to FIG. 1A is used for the experiments. The fluidic device is maintained at a fixed temperature in a thermostat chamber and is connected to 3 bottles containing buffer, a conditioning solution (e.g. proteins) and a bacterial suspension from which liquids are injected through the system by a peristaltic pump. All the liquids are then channeled to a waste collecting bottle.

The whole system is mounted at least two hours before the beginning of the experiment. The removable surface inside the flow cell is a glass cover slip (VWR, 24×60 mm) on which a layer of PDMS (Dow Corning, Sylgard 184) is spun cast using a spincoater. Therefore, PDMS is dissolved in 5 parts of hexane and deposited on the glass surface, the latter is thus spun around at 3000 RPM for 1 min. Coated slides are then cured at 95° C. for 1 hour. The prepared surfaces are used to close the flow chamber and sealed by the means of plates.

The fluidic device and bottles are fitted with tubings (Fischer Scientific Silicone tubings 1.6 mm inner diameter, 3.2 mm outer diameter), connectors (Kynar), and equipped with air filters made of PTFE (porosity 0.22 μm).

The whole apparatus is autoclaved (121° C., 15 min, 1.5 bar) before being used. PBS is the only liquid that is autoclaved with the system. The apparatus is left to dry at 50° C. for an hour and subsequently to cool down at room temperature for 20 minutes. The bottles are then kept in a water bath to ensure that the liquids pumped through the flow cell are at the desired temperature.

At the beginning of the experiment, conditioning solution and bacterial suspension and filtered in the appropriate bottles.

### E. Experiments

The adhesion of the two strains ATCC 12228 and ATCC 35984 has been tested on uncoated PDMS and on PDMS coated with diluted bovine serum and TSB. Each combination has been repeated 3 times, a total of 18 experiments.

Practically, the order in which liquids is injected in the flow cell are listed in Table 1. All steps are carried out at a constant flow rate of 0.5 ml/min corresponding to a hydraulic retention time (HRT) of 1 minute.

TABLE 1
Order of liquids injection in the flow cell device step by step

| Step | Type of liquid | Time |
|---|---|---|
| Filling up | PBS | 20 min |
| Coating of PDMS | Conditioning solution | 20 min |
| Rinsing | PBS | 10 min |
| Bacterial adhesion | Bacterial suspension | 20 min |
| Rinsing | PBS | 45 min |

For each experiment, the bacterial charge in the suspension is estimated by serial dilution and plating on non-selective growth medium. For quantification, the flow cells are transferred on an inverted microscope (Leica, DMI 6000) to image the surface of PDMS with a 40× objective (PH2, long working distance, Leica). In total, 21 pictures were taken for each experiment, in 3 series of 7 pictures along the whole distance of the chamber. ImageJ, an open-source software designed for image treatment, is used to detect the number of adherent bacteria per mm².

The bacterial counts on the surfaces are standardized according to the bacterial charge in bacterial suspensions. The adjusted data were then analyzed in JMP (statistical software) in two different ways. First, the variance is quantified assuming all the effects (strain, surface, distance, series, and the interactions between each of them) were random effects. This type of algorithm determines where the variability of the whole experimental plan is coming from. The results of this analysis are listed in Table 2.

TABLE 2
Results of the analysis of variance in JMP based on the results of 6
adhesion experiments (in triplicate). The percentage refers to the part
of variability in the results attributed to a particular parameter.

| Parameters | Percentage (%) |
|---|---|
| Surface | 60.43 |
| Strain | 19.66 |
| Residues | 13.43 |
| Distance | 4.62 |
| Surface * Strain | 1.64 |
| Series | 0.23 |
| Total | 100.00 |

Secondly, a mixed model is used to calculate mean number of adherent bacteria/mm² for each strain on every type of surfaces. To do so, surface, strain and the interaction surface*strain are considered as fixed parameters (meaning they are of interest to this study), the rest of the effects are set as random, implying their effect on the results is of no interest to the analysis performed here. The mixed model implement here is based on the same basic principle as a regression:

y=βX+μZ+ε

In this equation, y is a vector of observations (results) that is explained by a vector of fixed effects (β), a vector of random effects (ρ) (both are modulated by a matrix of regression parameters, respectively X and Z) and finally a term grouping random errors ε.

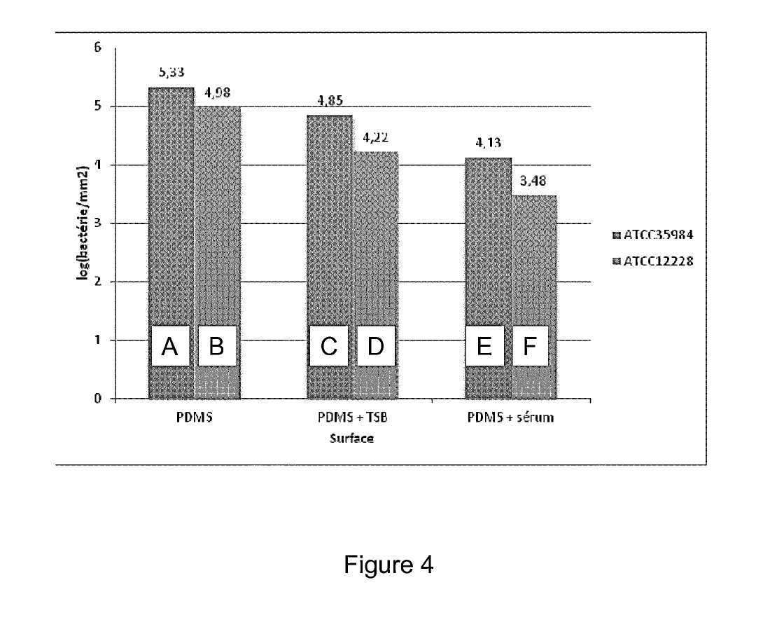

Using this model, a Tukey HSD test (Honestly Significant Differences) is performed, a conservator statistical tool to determine which mean adhesion values are different from one another. This test is chosen because it reduces the risk to consider two values different when they are in fact not. The final results are shown in FIG. 4, illustrating the mean adhesion values of ATCC 12228 and ATCC 35984 on PDMS surfaces either bare (A and B), coated with TSB (C and D) or with serum (E and F) expressed as the log 10 of adherent bacteria/mm². A is significantly different from B, C and E. B is significantly different from D and F. C is significantly different from D and E is significantly different from F.

Two main observations can be made based on these results. Firstly, on the three tested surfaces, ATCC 35984 proved to adhere more efficiently than ATCC 12228, with larger differences in the case of coated surfaces. Secondly, the physico-chemical properties of the surface have a significant impact on S. epidermidis adhesion, characterized by up to 1.5 log reduction if PDMS is coated with diluted bovine serum. Using the fluidic device according to the present invention detailed analysis can be done studying the adherence of different bacterial strains to different types of substrates.

### Example 3

In the present example, the fluidic device according to the present invention is used to study the adsorption of proteins from different solutions onto PDMS under different conditions (laminar flow and low shear rate).

### A. Conditioning Solutions

Several conditioning solutions are used in this study: undiluted adult bovine serum, 10 fold, 100 fold and 1000 fold diluted adult bovine serum, 1 g/L bovine serum albumin (BSA), 0.5 g/L BSA and 0.5 g/L human fibrinogen, Trypicase Soy Broth (TSB). Diluted bovine serum is obtained by diluting adult bovine serum (Sigma) in PBS to obtain 10, 100 and 1000 fold dilutions. 1 g/L BSA and 0.5 g/L BSA+0.5 g/L human fibrinogen are obtained by weighing crystallized powders and solubilizing in PBS. TSB is prepared by diluting 30 g of powder (Biorad) in 1 L of distilled water. All solutions except TSB are prepared just before use and filter sterilized.

### B. Fluidic Device

The fluidic device according to FIG. 1A is used for the experiments. The fluidic device is maintained at a fixed temperature in a thermostat chamber and is connected to 2 bottles containing buffer and a conditioning solution (e.g. proteins) from which liquids are injected through the system by a peristaltic pump. All the liquids are then channeled to a waste collecting bottle.

### C. Adhesion Experiments

Practically, the order in which liquids are injected in the flow cell is listed in Table 3. All steps were carried out at a constant flow rate of 0.5 ml/min corresponding to a hydraulic retention time (HRT) of 1 min.

TABLE 3
Order of liquids injection in the flow cell device step by step

| Step | Type of liquid | Time |
|---|---|---|
| Filling up | DI water | 20 min |
| Coating of PDMS | Conditioning solution | 20 min |
| Rinsing | DI water | 45 min |

All solutions in section A have been tested twice according to the protocol described here above. The repetitions have been carried out on different days.

The analysis of the surface atomic composition was done using XPS, a powerful technique to quantitatively and qualitatively determine surface atomic composition. Therefore, X-rays are aimed at the surface, and absorbed by electrons from the atom cores that are expelled with a defined kinetic energy. The formula expressing how the energy from the X-rays is dissipated is shown here below:

E_{xrays}=E_{binding}+E_{kinetic}+E_{residual}

E_{kinetic} is measured by the machine when photoelectrons hit the detector. E_{residual} comprises energy losses by the electron in its travel from the atom core to the detector of the machine. These are usually very low and can be due to non-elastic collisions with other particles in the surface or in the air. As a result, E_{binding} is the resulting measure given by the apparatus. To experimentally proceed to the analysis, flow cells are dismounted to recover the PDMS slide. The latter is immediately stored into DI water before analysis. The slide is dried with a gentle N₂ flow and broken into 1 cm² square pieces in order to be inserted into the XPS. Samples are inserted in the XPS at least 16 hours before the start of the experiment in order to apply vacuum around the sample. The analysis is operated in two phases. First, the machine records a general spectrum, collecting electrons from a large range of kinetic energy (i.e. coming from all types of atoms present on the surface). Each peak detected in the general spectrum corresponds to an atom. A quantification of the surface abundance of each atom is computed. Hence, a more precise analysis is then run on each of the detected peaks. The machine performs a more intense illumination and collects photoelectrons in a narrow range of kinetic energy for a certain time lapse. This results in a detailed spectrum showing more distinct and isolated energy peaks which can be associated with a particular state of binding of the Carbon atom, respectively C bound to C or Si, C bound to O, or N and C involved in peptide bond or carboxylic acid. Intensity in counts/s is directly proportional to the quantity of each Carbon type on the surface. By calculating the ratio between the quantity of N₁ₛ and C₁ₛ involved in the peptide bond an assessment can be made as to the amount of proteins (a ratio is close to 1 indicates that the nitrogen on the surface is a protein). The analyzed data can be displayed as shown in Table 4 here below.

TABLE 4
Data computed from the spectrum of PDMS coated with 100% serum, atomic percentage
and relative abundance of C functions are displayed in row 2. N/(C═O, O—C—O)
ratio close to 1 expresses that nitrogen is mainly originating from proteins

| | Atomic and functional composition (%) | | | | | | | Ratio |
|---|---|---|---|---|---|---|---|---|
| | C═O, | | C—Si, | | | | | N/(C═O, |
| Sample | O—C—O | C—(O, N) | C—(C, H) | C total | O | N | Si | O—C—O) |
| Serum 100% | 5.3 | 8.2 | 40.2 | 53.6 | 21.0 | 4.9 | 20.5 | 0.9 |
| on PDMS | | | | | | | | |

First, control PDMS surfaces (PDMS-1 and PDMS-2) on which only PBS has been injected have been analyzed in order to check for contamination. The results are shown in Table 5.

TABLE 5
Atomic and functional quantification by XPS of PDMS surfaces

| | Atomic composition (%) | | |
|---|---|---|---|
| Sample | C | O | Si |
| PDMS theory | 50 | 25 | 25 |
| PDMS-1 | 45.9 | 22.5 | 31.6 |
| PDMS-2 | 46.9 | 23.6 | 29.5 |

This shows that the surfaces of PDMS display a stable chemical composition, although differing slightly from the theoretical composition of PDMS.

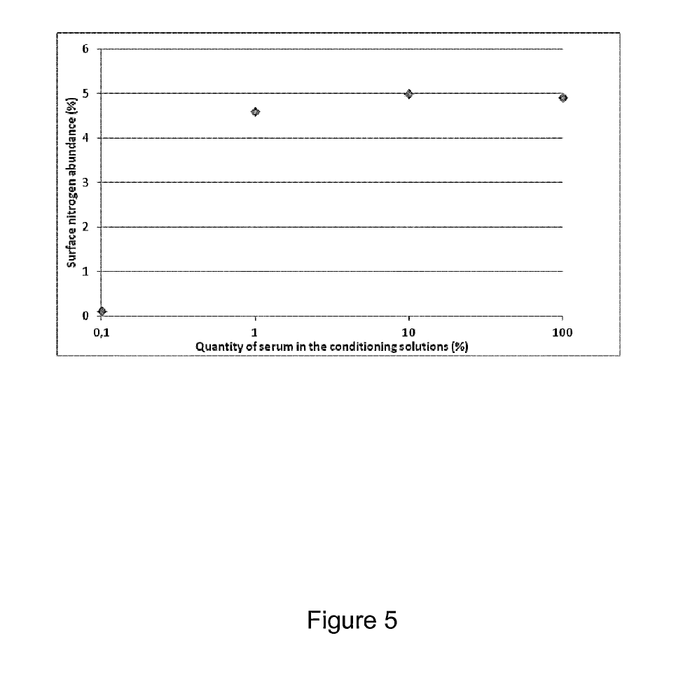

Subsequently, various dilutions of adult bovine serum were used to condition PDMS surfaces. This has been performed in order to determine the saturation point of the surface with proteins. Table 6 shows the treated XPS results in terms of atomic concentration and FIG. 5 plots the nitrogen surface percentage values of PDMS surfaces coated with dilutions up to 1000 times of adult bovine serum.

TABLE 6
Atomic and functional quantification by XPS of PDMS surface
coated with several dilutions of bovine serum in PBS

| | Atomic and functional composition (%) | | | | | | | | Ratio |
|---|---|---|---|---|---|---|---|---|---|
| | C═O, | | C—Si, | | | | | | N/(C═O, |
| Sample | O—C—O | C—(O, N) | C—(C, H) | C total | O | N | Si | F | O—C—O) |
| Serum 100% | 5.3 | 8.2 | 40.2 | 53.6 | 21.0 | 4.9 | 20.5 | — | 0.9 |
| on PDMS | | | | | | | | | |
| Serum 10% | 5.2 | 8.0 | 40.9 | 54.1 | 21.2 | 5.1 | 19.6 | — | 1.0 |
| on PDMS-1 | | | | | | | | | |
| Serum 10% | 5.9 | 9.9 | 38.2 | 54.1 | 20.7 | 5.3 | 19.9 | — | 0.9 |
| on PDMS-2 | | | | | | | | | |
| Serum 1% | 4.8 | 7.8 | 40.4 | 52.9 | 21.0 | 4.6 | 21.1 | 0.4 | 1 |
| on PDMS | | | | | | | | | |
| Serum 0.1% | u.d.l. | u.d.l. | 46.5 | 46.5 | 23.2 | 0.1 | 30.1 | — | N.D. |
| on PDMS | | | | | | | | | |

—: not detected
N.D.: not determined
u.d.l.: under detection limit

FIG. 5 shows that a plateau appears from the 10 fold dilution. Coherently with the theory on protein adsorption, maximal protein adsorption on a surface is achieved when a monolayer is formed. The saturation is dependent on time of exposure of the surface to proteins, concentration of the latter, temperature, surface roughness, etc. In this case, only the protein concentration is variable, all other factors are fixed. 10 fold dilution of serum was chosen for the rest of the experiments.

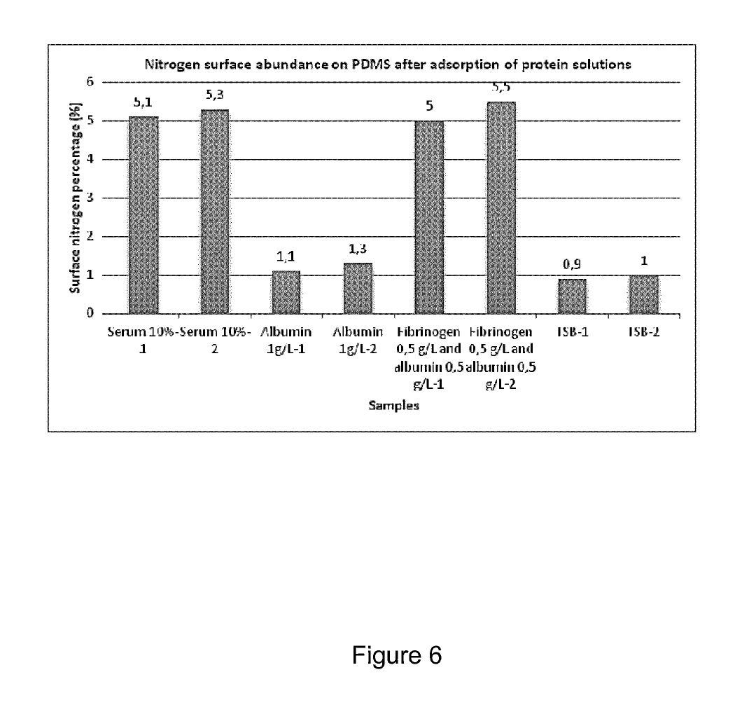

The samples conditioned by 1 g/L BSA, 0.5 g/L BSA and 0.5 g/L human fibrinogen and TSB can be compared to PDMS coated with 10 fold diluted bovine serum with respect to the percentage of nitrogen present on the their surface. For all these samples, N₁ₛ/C₁ₛ (O—C—O, C═O) are equal or close to 1, indicating that nitrogen on the surface is in the form of proteins. The nitrogen abundance values are summarized in FIG. 6.

FIG. 6 shows a plot of the values of nitrogen abundance on the surface conditioned PDMS. The repetitions for each sample have been plotted on the graph and marked as 1 and 2. The graph indicates that only human fibrinogen mixed with BSA can reach the value of 5% of adsorbed nitrogen (i.e. assumed threshold for a dense protein monolayer on PDMS in these flow conditions). BSA alone displays 5 times less nitrogen then the serum reference. Although BSA efficiently adsorbs on surfaces, it seems to form a less dense layer on PDMS under laminar flow. Also, these results show that, in competition with fibrinogen, BSA might be largely displaced from the surface by fibrinogen. Indeed, the latter is larger but has a higher affinity for the surface. Finally, TSB occurs to be the least adsorbent solution on PDMS with nitrogen content left on PDMS just below 1%. TSB is majorly composed of peptides derived from the lytic digestion of soy proteins and casein by papain and trypsin. Smaller peptides are known to have a weaker affinity for surface than larger proteins.

Additionally the contact angle of the surface with water has been measured for each sample. Therefore, a 0.3 μL drop of DI water is deposited on a N₂ dried sample and the behavior of the droplet on the surface is monitored by a camera and edge detection software. The angle between the surface and the droplet is measured 5 s after the initial contact. Each surface has been tested at least 5 times. The contact angle of water on the solid surfaces is represented as Θ and is assessed by edge detection software. If Θ<90° the surface is considered globally hydrophilic, when Θ>90° the surface is considered as hydrophobic.

Every sample that was analyzed in XPS has also been submitted to a water contact angle measurement. The results of the experiments are shown in Table 7.

TABLE 7
Water contact angle measurements on PDMS surfaces coated with
diluted bovine serum, BSA, BSA + fibrinogen, and TSB.

| Sample | Water contact angle θ (°) |
|---|---|
| PDMS-1 | 115.4 ± 1.4 (N = 10) |
| PDMS-2 | 114.9 ± 0.7 (N = 12) |
| PBS on PDMS-2 | 114.9 ± 0.7 (N = 9) |
| Serum 10% on PDMS-1 | 100.5 ± 3.1 (N = 7) |
| Serum 10% on PDMS-2 | 99.1 ± 2.2 (N = 13) |
| Serum 1% on PDMS | 109.5 ± 1.7 (N = 9) |
| Serum 0.1% on PDMS | 112.2 ± 1.4 (N = 7) |
| Albumine on PDMS-1 | 109.8 ± 1.6 (N = 8) |
| Albumine on PDMS-2 | 113.6 ± 0.5 (N = 9) |
| Fibrinogen and BSA on PDMS-1 | 109.6 ± 1.9 (N = 7) |
| Fibrinogen and BSA on PDMS-2 | 108.5 ± 0.7 (N = 9) |
| TSB on PDMS-1 | 111.7 ± 0.2 (N = 5) |
| TSB on PDMS-2 | 112.6 ± 0.9 (N = 13) |

Using the fluidic device according to the present invention detailed analysis can be done studying the adsorption of proteins from different solutions onto PDMS under different conditions.

### Example 4

In order to study the effect of a specific compound on the growth of a bacterial strain developing as a biofilm on a substrate (membrane) in particular flow conditions the fluidic system according to the present invention can be used. In particular the system according to FIG. 7 can be used. The fluidic device is incorporated into a fluid flow system (FIG. 1A) where liquid is flown through the fluidic device using a pumping system. The fluidic device comprises a two fluidic chamber (2) each having a separate inlet (3), outlet (4), inlet flow distributor (6) and outlet flow distributor (9). The fluidic chambers are positioned adjacent to each other and are separated by detachable and/or removable surface (10).

For the present example a 0.45 μm porosity membrane made of polyethylene (PE) is used as detachable and/or removable surface. The device is autoclaved for sterility prior to use.

The two fluidic chambers are used in parallel, one is used as a control experiment, and the other is used to test the effect of the compound of interest.

In the first fluidic chamber an inoculum of a bacterial strain in a stationary phase culture is fed for an hour. Then, fresh growth medium is continuously injected for 72 h and discarded in a waste bottle as it goes out through the outlet. In the second fluidic chamber, 200 ml of a solution either containing the biologically active compound or without the latter (for the control experiment) is circulating in a loop for 72 h.

The flow in both channels is set to the same rate. The membrane selectively allows the compound circulated through the second fluidic chamber to diffuse into the first fluidic chamber but prevents bacteria growing on the membrane surface to cross to the second fluidic chamber.

At the end of the experiment, the membrane is recovered by dismounting the setup and the number of adherent bacteria on the membrane is determined.

It was observed that the amount of viable cells on the membrane surface was 3 logs higher in the control experiment compared to the membrane submitted to the compound of interest.

The compound reduced the ability of bacterial cells to develop on the surface of the membrane as a biofilm.

### Example 5

In order to simulate a sophisticated in vitro intestinal barrier in between two flows (mimicking blood flux and gut lumen respectively) the fluidic system according to the present invention can be used. In particular the system according to FIG. 7 can be used. The fluidic device can for instance be used to study exchanges supported by enterocytes.

A membrane of 5 μm porosity polycarbonate (PC) is used as detachable and/or removable surface for this experiment. The membrane supports the growth of enterocytes cell lines in McCoy's 5A modified medium supplemented with 10% fetal bovine serum, 100 U/mL penicillin, and 100 μg/mL streptomycin.

A cell suspension is injected in the first fluidic chamber and at the same time, a flow of supplemented McCoy's 5A is injected in the second fluidic chamber. Both flows are stopped and the cells are left to sediment on the PC membrane for an hour. The flow in both fluidic chambers are resumed for a period of 15 days with fetal bovine serum circulating in the second fluidic chamber instead of McCoy's 5A at a very low flow rate (0.1 ml/min) to allow the cells to completely cover the membrane surface.

Once a layer of cells is obtained, the fluids injected in both fluidic chambers can be changed and be injected in loop mode (outlet leads to a recipient from which the liquid is pumped towards inlet). The goal is to witness an active transfer mediated by epithelial cells of some compounds comprised in fluid of the first fluidic chamber to fluid in the second fluidic chamber. Compound concentrations can be assessed by HPLC by picking samples from the outlet of both fluidic chambers.

1. A fluidic device for the analysis of a surface comprising:
a fluidic chamber comprising one or more surfaces enclosing the fluidic chamber through which fluid can flow along a flow path, wherein at least part of said fluidic chamber is casted with a polymeric material characterized by having a Young's Modulus [E] ranging between 500 kPa and 5 MPa;
an inlet port fluidically connected to said fluidic chamber through an inlet flow distributor for distributing the incoming fluidic flow from said narrow inlet port to said wider fluidic chamber;
an outlet port fluidically connected to said fluidic chamber optionally through an outlet flow distributor for distributing the outflowing fluidic flow from said wider fluidic chamber to said narrow outlet port;
characterized therein that said fluidic chamber comprises at least one detachable and/or removable surface for analysis purposes.

2. A fluidic device according to claim 1, wherein at least part of said fluidic chamber is made from a polymeric material having Young's Modulus [E] ranging between 500 kPa and 2 MPa.

3. A fluidic device according to claim 1, wherein at least part of said fluidic chamber is made from a polymeric material chosen from PDMS, acrylate elastomers, fluoroelastomers and styrenic based elastomers.

4. A fluidic device according to claim 1, wherein said fluidic chamber comprises a bottom surface, two side surfaces and a top surface, wherein at least part of said top or bottom surface are detachable and/or removable.

5. A fluidic device according to claim 1, wherein at least part of said fluidic chamber is transparent for optical imaging, for microscopy, and/or for fluorescence imaging, thereby providing an imaging observation site.

6. A fluidic device according to claim 1, further comprising observation zones and/or sensors located on or in one or more surface enclosing said fluidic chamber, for observing and/or monitoring the formation and/or cultivation of the surface-dwelling multicellular layer on said detachable and/or removable surface.

7. A fluidic device according to claim 1, wherein said detachable and/or removable surface is made from or provided with an adherent surface material suitable for adhering a surface-dwelling multicellular layer, wherein said surface material models a surface likely to be involved in cell adhesion and/or formation of the multicellular layer.

8. A fluidic device according to claim 1, wherein said adherent surface material is chosen from the group comprising aluminum, stainless steel, silver, copper, hydroaxyapatite, silicon, latex, urethane, PVC, ceramic, steel, gold, titanium, polyethylene, polysiloxanes, biocompatible glasses, poly-methylmethacrylate, Teflon (or PTFE), polypropylene, polystyrene, polyamides, polyethers, polyesters, coated block polymers of polyethylene oxide (PEO), polypropylene oxide (PPO), polybutylene oxide (PBO), hydrogels, food film polymers, polycarbonate filters and minerals.

9. A fluidic device according to claim 1, wherein said inlet flow distributor or part thereof slopes downward relative to the horizontal position of the fluidic chamber.

10. A fluidic device according to claim 1, wherein said inlet flow distributor comprises:
a first distributor region shaped as an isosceles trapezoid;
a second rectangular shaped distributor region;
wherein said first distributor region is fluidically connected to the narrow inlet channel through the smaller of the two parallel sides and fluidically connected to said second rectangular shaped distribution region through the larger of the two parallel sides of said isosceles trapezoid, wherein said second rectangular shaped distribution region is further fluidically connected to said fluidic chamber,
characterized therein that said second distribution region slopes downward relative to the horizontal position of said fluidic channel.

11. A fluidic device according to claim 1, wherein the depth of the fluidic chamber ranges between 0.1 and 5 mm.

12. A fluidic device according to claim 1, wherein said device comprises at least two fluidic chambers with corresponding inlet and outlet ports and distributors, wherein said two fluidic chambers are arranged adjacent to each other and are separated by detachable and/or removable surface, wherein said detachable and/or removable surface is preferably a membrane.

13. A method for performing an analysis between a surface of interest and a fluid of interest, comprising:
a) providing a fluidic device comprising:
a fluidic chamber comprising one or more surfaces enclosing the fluidic chamber through which fluid can flow along a flow path, said fluidic chamber comprising at least one detachable and/or removable surface of interest;
an inlet port fluidically connected to said fluidic chamber;
an outlet port fluidically connected to said fluidic chamber;
b) dispensing fluid of interest through said fluidic device, thereby contacting the fluid of interest with the surface of interest; and
c) monitoring interaction between the surface of interest and a fluid of interest under varying conditions.

14. A method according to claim 13, for cultivating and monitoring surface-dwelling multicellular layers, comprising:
a) providing a fluidic device comprising:
a fluidic chamber comprising one or more surfaces enclosing the fluidic chamber through which fluid can flow along a flow path, said fluidic chamber comprising at least one detachable and/or removable surface of interest for the cultivating multicellular layers;
an inlet port fluidically connected to said fluidic chamber;
an outlet port fluidically connected to said fluidic chamber;
b) dispensing a fluid of interest through said fluidic device, said fluid of interest being a liquid growth medium, said liquid growth medium optionally comprising microorganisms, animal cells, plant cells or fungi cells, into said fluidic chamber, said growth medium flowing across said detachable and/or removable surface, thereby generating a surface-dwelling multicellular layer on said detachable and/or removable surface; and
c) monitoring the multicellular layer on said detachable and/or removable surface under varying conditions.

15. Method according to claim 13, wherein said varying conditions comprise different types of fluid media, different fluid flow rates, different temperatures, different compounds and/or combinations thereof.
